(19) United States
(12) Patent Application Publication
(10) Pub. No.: US 2002/0115059 A1
(43) Pub. Date: Aug. 22, 2002

(54) Drug screening system

(76) Inventors: Naohiro Terada, Gainesville, FL (US); Takashi Hamazaki, Gainesville, FL (US)

Correspondence Address: Stanley A. Kim, Akerman, Senterfitt & Eidson, P.A., 222 Lakeview Avenue, Suite 400, P.O. Box 3188, West Palm Beach, FL, 33402-3188, US

(21) Appl. No.: 10/045,721

(22) Filed: Oct. 26, 2001

Related U.S. Application Data
(60) Provisional application No. 60/243,549, filed on Oct. 26, 2000.

(51) Int. Cl. C12Q 1/00; G01N 33/53; G01N 33/567; C12N 5/06

(52) U.S. Cl. 435/4; 435/7.2; 435/7.21; 435/354

(57) ABSTRACT

A method for identifying a drug candidate for promoting tissue-specific differentiation of a stem cell includes the steps of: providing a library of test substances and an in vitro culture of stem cells divided into at least two subcultures; contacting one of the subcultures with the first test substance from the library and a second subculture with a second test substance from the library; culturing the subcultures under conditions that would promote tissue-specific differentiation of the stem cells if an agent that promoted tissue-specific differentiation was in contact with the stem cells; and analyzing the cells in the subcultures for increased tissue specific gene expression.

### CROSS REFERENCE TO RELATED APPLICATION

The present application claims the priority of U.S. provisional patent application No. 60/243,549 filed Oct. 26, 2000.

### STATEMENT AS TO FEDERALLY-SPONSORED RESEARCH

This invention was made with United States government support under grant number DK59699 awarded by the National Institutes of Health. The United States government may have certain rights in the invention.

### FIELD OF THE INVENTION

The invention relates generally to the fields of biology, pharmaceuticals and medicine. More particularly, the invention relates to drug screening systems.

### BACKGROUND

In order to identify potentially useful drugs from a large library of chemical compounds, pharmaceutical companies employ various different types of screening assays. Among these, in vitro screening assays utilize cultures of cells to identify compounds that alter such cells' physiology. For example, in order to identify potential anti-cancer drugs, cultured cancer cells can be contacted with a library of different compounds. Those compounds that kill the cells or stop their growth are potential anti-cancer drugs.

In the search for candidate drugs, an ideal in vitro screening assay should closely mimic the condition to be treated. In addition such assays should be accurate (repeatable with similar results), rapid, adaptable for high-throughput, and low in cost.

### SUMMARY

The invention relates to the development of a new system for screening for drug candidates that are potentially useful for promoting tissue-specific differentiation. The system employs in vitro-cultured embryonic stem (ES) cells to screen chemical compounds for their ability to promote tissue-specific differentiation. Those that induce the differentiation of ES cells towards a specific tissue lineage are considered drug candidates that can be further tested (e.g., in in vivo animal-based assays) to identify drugs useful for regeneration of lost or damaged organs.

Accordingly, the invention features a method for identifying a drug candidate for promoting tissue-specific differentiation of a stem cell. This method includes the steps of: (A) providing a library of test substances made up of least two test substances having different molecular structures; (B) providing an in vitro culture of stem cells, the culture being divided into at least two subcultures; (C) contacting one of the subcultures with one of the test substances and another of the subcultures with another test substance; (D) culturing the subcultures under conditions that would promote tissue-specific differentiation of the stem cells if an agent that promoted tissue-specific differentiation was in contact with the stem cells; and (E) analyzing the cells in the subcultures for increased tissue-specific gene expression (e.g., as measured by increased tissue-specific mRNA expression). The presence of increased expression of a given tissue-specific mRNA in the cells of a particular subculture indicates that the substance added to that particular subculture is a drug candidate for promoting the tissue-specific differentiation of a stem cell

The stem cells employed in the method can be embryonic stem cells such as mammalian embryonic stems cells, e.g., murine embryonic stems cells (for instance, R1 cells) or human embryonic stems cells.

The conditions that would promote tissue-specific differentiation of the stem cells can include culturing the subcultures in a differentiating medium; at about 37° C.; in a humidified, carbon-dioxide containing incubator; and/or for a time period of at least five days (e.g., at least seven days, or between seven and eighteen days).

In one variation of the method of the invention, the subcultures are cultured in a microtiter plate.

The step of analyzing the cells in the subcultures for increased tissue-specific gene expression can include isolating mRNA or total cellular RNA from the subcultures. This step can also include reverse-transcribing the mRNA to create cDNA.

Analyzing the cells in the subcultures for increased tissue-specific gene expression can involve a polymerase chain reaction (PCR), immobilizing the isolated mRNA on a substrate (and, optionally, contacting the substrate with a probe that specifically hybridizes to the tissue-specific mRNA), and/or using gene chip technology.

As used herein, the phrase “tissue-specific gene expression” is meant that a gene or particular isoform of a gene (e.g., as measured by mRNA or protein levels) is exclusively expressed in a particular cell type. This phrase can also mean that a gene or particular isoform of a gene is expressed at a sufficiently higher level in one particular cell type than in another cell type such that the two cell types can be distinguished on this basis.

The phrase “stem cell,” as used herein, means any cell having the potential to differentiate into at least two different cell types. For example, a hematopoietic stem cell has the potential to differentiate into a lymphocyte as well as an erythrocyte. As used herein, an “embryonic stem cell” is a stem cell derived from an embryo. Typically, an embryonic stem cell is considered “totipotent” in that it is capable of giving rise to all types of differentiated cells found in the organism from which it was derived. A “pluripotent stem cell” generally refers to a stem cell capable of differentiating into several different finally differentiated cell types. Pluripotent stem cells are typically less than totipotent.

By “library of test substances” is meant any compilation of two or more different molecules (e.g., organic compounds, inorganic molecules, nucleic acids, polypeptides, etc.). Generally, a library of test substances includes at least 100 (e.g., 200; 500; 1,000; 5,000; 10,000; 50,000; 100,000; 500,000; 1,000,000 or more) different molecules each distinguishable by molecular structure.

The term “subculture” is used herein to refer to any culture derived from another culture. For example, a culture of stems cells can be divided into two or more different aliquots of stems cells. Each different aliquot is a subculture of the original culture of stem cells.

By the phrase “differentiating medium” is meant any tissue culture medium that can promote (or at least not prevent) differentiation of stem cells.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including any definitions will control. In addition, the particular embodiments discussed below are illustrative only and not intended to be limiting.

Other features and advantages of the invention will be apparent from the following detailed description, and from the claims.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a flow chart outlining a preferred screening system of the invention.

FIG. 2 is a schematic overview of an in vitro differentiation protocol of the invention. Acidic FGF (100 ng/ml) was added between Days 9 and 12 as an early-stage factor for hepatic maturation. HGF (20 ng/ml) was added between Days 12 and 18 as a mid-stage factor. Oncostatin M (10 ng/ml), dexamethasone (10⁻⁷M), and ITS mixture (5 mg/ml insulin, 5 mg/ml transferrin, 5 μg/ml selenious acid) were added as late stage factors between Days 15 and 18.

FIG. 3 is a blot showing expression of endodermal and hepatic differentiation marker genes in differentiating ES cells. ES cells were cultured under the protocol shown in FIG. 2 without using a collagen-coated dish or additional growth factors for hepatic maturation. Cells were harvested at the indicated time (days). mRNA expression of transthyretin (TTR), alpha-fetoprotein (AFP), alpha 1-antitrypsin (AAT), albumin (ALB), glucose-6-phosphatase (G6P), tyrosine transaminase (TAT), and β-actin was examined by reverse transcriptase-polymerase chain reaction (RT-PCR). Molecular size marker for DNA (M), fetal liver at embryonic day 12 (FL), adult liver at 3 weeks old (AL). Results shown here and below are representative of three to five independent experiments.

FIG. 4 is a blot showing the effects of a collagen-coated dish and addition of growth factors on expression of late hepatic differentiation markers. ES cells were cultured under the protocol shown in FIG. 2 with or without using a collagen-coated dish, in the absence or presence of growth factors (GFs) for hepatic maturation. Cells were harvested at Day 18, and mRNA expression of ALB, G6P, TAT and β-actin was examined by RT-PCR. Fetal liver at embryonic day 12 (FL), adult liver at 3 weeks old (AL).

FIG. 5 is a blot showing the effects of early, mid and late stage factors on expression of late hepatic differentiation markers. ES cells were cultured under the protocol shown in FIG. 2. a) No growth factor (none), early-stage factor alone (early), mid-stage factor alone (mid), late-stage factors alone (late), mid- and late-stage factors (mid/late), or early-, mid- and late-stage factors (early/mid/late) were added as growth factors for hepatic maturation. b) No growth factor (none), oncostatin M, dexamethasone and ITS mixture (OSM/Dex/ITS), oncostatin M (OSM), dexamethasone (Dex), or ITS mixture (ITS) were added as late growth factors for hepatic maturation. No early or mid factors were added. Cells were harvested at Day 18, and mRNA expression of G6P, TAT, and β-actin was examined by RT-PCR.

FIG. 6 is a blot showing the effects of SEK1 knockout on expression of late hepatic differentiation markers. SEK1 null ES cells (SEK1 −/−) and their parental ES cells (SEK1+/+) were cultured under the protocol shown in FIG. 2. a) SEK1 expression. Cells were harvested at Day 18, and expression of SEK1 mRNA was examined using RT-PCR. b) Induced expression of G6P and TAT. No growth factor (none), late-stage factors alone (late), mid and late stage factors (mid/late), or early, mid and late stage factors (early/mid/late) were added as growth factors for hepatic maturation. Cells were harvested at Day 18, and mRNA expression of G6P, TAT, and β-actin was examined by RT-PCR. c) JNK activity. Late stage factors were added into differentiating ES cells at Day 15. Cells were harvested at the indicated time after addition of the factors (0, 15 or 30 min later). As a control, cells were irradiated by ultra-violet for 15 min. JNK activity in the cell lysates was measured as described in Example 2.

FIG. 7 is a series of histograms of a flow cytometric analysis of ES cells. GF(+)-a combination of growth factors was added to ES cells expressing green fluorescent protein under the control of an alpha-fetoprotein promoter. Control- ES cells expressing green fluorescent protein under the control of an alpha-fetoprotein promoter were not contacted with growth factors. GFP(−) ES Cells not expressing green fluorescent protein.

### DETAILED DESCRIPTION

This invention encompasses a system for screening for drug candidates that are potentially useful for promoting tissue-specific differentiation. The below-described preferred embodiments illustrate adaptations of this system. Nonetheless, from the description of these embodiments, other aspects of the invention can be made and/or practiced based on the description provided below.

General Methods

Methods involving conventional molecular biology techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990. PCR-primer pairs can be derived from known sequences by known techniques such as using computer programs intended for that purpose (e.g., Primer, Version 0.5, ©1991, Whitehead Institute for Biomedical Research, Cambridge, Mass.). Methods of preparing libraries and of drug screening are described in Smith, C. G., The Process Of New Drug Development, CRC Press, 1992; and Advances in Drug Discovery Techniques, ed. Alan L. Harvey, John Wiley & Sons, 1998.

Identifying Drug Candidates

The invention provides a method for identifying a drug candidate for promoting tissue-specific differentiation of a stem cell. A preferred version of this method is performed by first providing (1) a library of test substances and (2) an in vitro culture of stem cells. The cultures of stem cells is then divided into two or more subcultures (e.g., 96 different subcultures in one 96 well culture plate). The library of test substances is also divided into subsets containing one or more (but not all) of the test substances in the library. Each subculture of stem cells is then contacted with one of the subsets of the test substances. Different subcultures of stems cells are contacted with different subsets of test substances so that each of the different subsets of test substances can be evaluated. The cells are then cultured under conditions that would promote tissue-specific differentiation of the stem cells if an agent that promoted tissue-specific differentiation was in contact with the stem cells. After culturing the cells for a sufficient period of time to induce physiological changes associated with differentiation, the cells are analyzed for increased tissue-specific gene expression (e.g., increased levels of a tissue-specific mRNA). Increased expression of a particular tissue specific mRNA in a particular subculture of cells indicates that the subset of test substances added to that subculture includes a drug candidate for promoting differentiation of a stem cell into a cell that particular tissue type.

For example, if a given subset of test substances induced an increase in a liver-specific mRNA (e.g., mRNA encoding albumin) in a subculture of stem cells, that subset of test substances would include a drug candidate for promoting differentiation of a stem cell into a liver cell (e.g., a hepatocyte). Similarly, if a another subset of test substances induced an increase in a cardiac muscle-specific mRNA (e.g., mRNA encoding alpha cardiac myosin-heavy chain) in a second subculture of stem cells, that subset of test substances would include a drug candidate for promoting differentiation of a stem cell into a cardiac muscle cell.

Library Of Test Substances

The invention utilizes a library of test substances that can be screened to identify drug candidates. The library can be composed of organic or inorganic chemical compounds such as those made by combinatorial chemistry techniques or traditional synthetic methods. Many pharmaceutical companies have chemical compound libraries that would be suitable for screening according the invention. For drug candidates, those organic or inorganic chemical compound libraries that are made up of small compounds (e.g., less than about 10,000; 5,000; 2,000, 1000, or 500 daltons) are preferred as smaller compounds often prove more suitable for delivery to a subject (or cell) and/or are non-immunogenic. Libraries useful in the invention might also be composed of proteins or peptides (including antibody and antibody fragments) or various nucleic acid molecules. Numerous methods of making such peptide/nucleic acid libraries are known.

Libraries of large numbers of substances can be utilized in the invention in several different ways. For example, a library can be separated into many different subsets with each subset containing substances of only one molecular structure and no two subsets containing the same substance. Each subset could then be added to a culture of stem cells for analysis. Alternatively, a library can be separated into many different subsets, each subset containing at least two (e.g, 2, 3, 4, 5, 10, 25, 50, 100, 1000, 10,000 or more) substances that differ from each other in molecular structure. In the latter method, those subsets that induce a desired response in a culture of stem cells can be selected for further analysis.

Stem Cells

Many different types of stems cells are known. A review of stem cells technology is provided in Petersen, B. E. and N. Terada, J. Am. Soc. Nephrol. 12:1773-1780. Any type of stem cell that is suitable for use in the method described herein might be used. Of particular interest for the invention are ES cells. ES cells are continuously growing stem cell lines of embryonic origin first isolated from the inner cell mass of blastocysts (Evans, M J. and Kaufman, M. H. (1981) Nature 292, 154-6; Martin, G. R. (1981) Proc. Natl. Acad. Sci. USA 78, 7634-8). ES cells are particularly preferred for use in the invention because of their capacity to be indefinitely maintained in an undifferentiated state in culture and their potential to develop into every cell type. ES cells can proliferate in vitro in an undifferentiated state on a feeder layer of mouse embryonic fibroblast cells (MEF) or in a medium containing leukemia inhibitory factor (LIF). The most rigorous test of the developmental potential of mouse ES cells is their ability to contribute to all cell lineages of chimeric animals-including the germ line (Bradley et al., (1984) Nature 309, 255-6).

In addition to their pluripotent ability to differentiate in vivo, ES cells can differentiate into multiple cell lineages in vitro as well. The in vitro differentiation of ES cells is induced by removing the ES cells from the feeder layer of MEF or by removing LIF from the culture medium, and then allowing them to form aggregates in suspension. ES cells aggregate into structures termed embryoid bodies (EB), in which all three germ layers develop and interact with each other. Well-differentiated EBs are composed of multiple differentiated cell types including neuronal, cardiac muscle, hematopoietic, and chondrocytic cells. EBs recapitulate many processes that take place during development (Doetschman et al., (1985) J. Embryol. Exp. Morphol. 87, 27-45).

For screening for drugs candidates applicable to mammals, mammalian stems cells are preferred. For example, rodent ES cell such as rat or murine ES cells (e.g., R1 embryonic stems cells available from Dr. Andras Nagy, Mount Sinai Hospital, Toronto, Canada) are utilized in the Examples described below. In a similar manner, primate (including human) ES cells (see, e.g., U.S. Pat. Nos. 5,843,780 and 6,200,806) might be used in the invention. Stem cells of other species, including those from: horse, cat, dog, sheep, cow, pig, guinea pig, chicken, rabbit, etc., might also be used.

Useful stem cells can be obtained and cultured as described herein or as previously reported. E.g., Evans M J, Kaufman M H, Nature 292:154-156, 1981; Martin G R: Proc Natl Acad Sci USA 78:7634-7638, 1981; Thomson et al., Science 282:1145-1147, 1998.

Contacting Stem Cells With Test Substances

Various methods of the invention include a step of contacting stem cells with test substances. For many substances, this can be accomplished simply by adding a desired amount of the substance to a subculture of stem cells. That is, a solid-phase substance to be screened is either dissolved in an appropriate solvent (e.g., water, saline or a buffered salt solution) and added to the culture. Alternatively, a solid phase substance to be screened can be directly added to the cultures. Liquid substances to be screened can also be delivered by direct addition to subculture of stem cells.

For applications where it is desirable to introduce substances into the interior (i.e., through the plasma membrane) of stems cells, any suitable known technique might be used. For example, for screening nucleic acid molecules (e.g., DNA or RNA) where it is desired to introduce the molecules into the cell interior, known techniques such as electroporation, gene gun technology, liposome-based methods, and calcium chloride-methods may be adapted for use in the invention. In an alternative variation of the invention, proteins, peptides, or small molecule compounds are immobilized on the stem cell culture dish (e.g., coated onto the wells of a 96 well plate). For delivery of insoluble or hydrophobic substances various surfactants and carriers might be used to help dissolve the substances. The latter should be carefully selected to avoid or minimize damage to the stem cells.

Culture Conditions That Promote Tissue-Specific Differentiation

To assess the effect of a substance on stem cell differentiation, a step of culturing the stem cells contacted with the substance can be employed. Suitable methods for culturing stem cells have previously been described. E.g., Evans M J, Kaufman M H, Nature 292:154-156, 1981; Martin G R: Proc Natl Acad Sci USA 78:7634-7638, 1981; Thomson et al., Science 282:1145-1147, 1998. A preferred method for culturing the stem cells contacted with the substance is also described below in Example 2.

In general, methods of culturing stem cells involve placing the cells in a tissue culture medium that would promote differentiation of the stem cells, if the cells were subjected to an appropriate stimulus. As a specific example, for murine ES cells, a medium of IMDM containing 2 mM L-glutamine, 100 units/ml penicillin, 100 μg/ml streptomycin (GIBCO BRL), 20% fetal bovine serum (Atlanta biologicals) and 300 μM monothioglycerol (Sigma) can be used as a differentiating medium. This medium does not include LIF as might be included in media used to maintain murine ES cells in an undifferentiated state. Another factor in promoting differentiation of the stem cells is the absence of feeder cells (e.g., murine fibroblasts) in the culture.

Other suitable culture conditions for promoting differentiation of stem cells include placing the cultures in a humidified, 5% carbon-dioxide containing incubator, maintaining the temperature at about 37° C. (e.g., between 35-39° C.) for murine or human stem cells. In general, after being contacted with the substances being screened, the subcultures are cultured under conditions that promote differentiation for about 7-14 days prior to being analyzed for modulation of gene expression. This time period may vary depending on the particular type of stem cells used and the particular differentiation pathway being analyzed. For example, in assays utilizing murine ES cells, for differentiation into cardiac myocytes, changes in gene expression may be analyzed before 8-10 days in culture. In comparison, for differentiation into hepatocytes, changes in gene expression may be analyzed before 15-18 days in culture.

Analyzing Cells For Increased Expression Of A Tissue-Specific mRNA

Methods within the invention include a step of analyzing a subculture of stem cells for increased tissue-specific gene expression, e.g., increased levels of a tissue-specific mRNA, increased mRNA stability, or increased mRNA expression.

Genes that are preferentially or exclusively expressed in particular tissue types are known. For example, genes that are preferentially or exclusively expressed within the nervous system include the following: Nova-1, Nova-2, N-type calcium channels, GABA(A) receptor, dopamine receptors, agrin, neurexins, synapsins, PPT, CaM, vacuolar H(+)-ATPase subunit B (isoform H057), renin, nestin, GFAP, and neurofilament H.

Genes that are preferentially or exclusively expressed within epithelia include E-cadherin and Estrogen receptor (ER)3. The gene flk1 is preferentially expressed in the vascular endothelium. Genes that are preferentially or exclusively expressed within the endoderm include the following: TTF1/Nkx2.1, Nkx2.6, Pax8, Pax9, Hex1, Hoxb1, Pdx1, Pax4, Pax6, Nkx2.2, Is1-1, NeuroD, cdx2, Hoxd genes, Pancreas amylase 2, Pancreas PDX-1, and Pancreas INSULIN.

Genes that are preferentially or exclusively, expressed within cardiac, skeletal and muscle tissue include the following: cartilage matrix protein, collagen II adult type, myotonin protein kinase gene, TEF-1, cardiac alpha actin© alpha actin), cardiac myosin heavy chain-alpha (MHC alpha), cardiac myosin heavy chain-beta (MHC beta), myosin light chain-1A (MLC1A), myosin light chain-1V (MLC1V), alpha-tropomyosin (alpha TM), cardiac troponin-T (Ctnt), atrial natriuretic factor (ANF), cytochrome C oxidase (COX) tissue-specific isoforms (VIa, VIIa, VIII), Hand1, FHL2, hCsx, calcitonin receptor-like protein, and aldosterone-synthase.

Genes that are preferentially or exclusively expressed within the pancreas, liver or prostate include the following: albumin, alpha-fetoprotein, alpha1-antitrypsin, pancreas amylase 2, pancreas PDX-1, pancreas INSULIN, hB1f (human B1-binding factor), kallikrein (KLK) gene clusters, apolipoprotein(a), plasminogen, insulin-like growth factor binding protein 1 (IGFBP-1), phenylalanine hydroxylase (PAH), S-adenosylmethionine synthetase (SAMS), transthyretin, tyrosine aminotransferase, glucose-6-phosphatase, dipeptidylpeptidaseIV, cytokeratin 19, biliary glycoprotein, gamma-glutamyltranspeptidase, vinculin, cytokeratin 18, cytokeratin 8, c-met, Gata-6, Gata-4, variant hepatocyte nuclear factor 1, hepatocyte nuclear factor1-alpha, hepatocyte nuclear factor4-alpha1, hepatocyte nuclear factor4-alpha7, hepatocyte nuclear factor3-alpha, hepatocyte nuclear factor3-beta, hepatocyte nuclear factor3-gamma, apolipoproteinB, Smad-4, evx-1, contrapsin, major urinary proteins, alpha-1-microglobulin/bikunin precursor gene, phosphoenolpyruvate carboxykinase, carbamoylphosphate synthetase I, inter-alpha 1-trypsin inhibitor, alpha 1 acid glycoprotein, haptoglobin, vitamin D-binding protein, ceruloplasmin, fibrinogen, alpha 2-macroglobulin, thiostatin, transferrin, and retinol-binding protein.

A gene that is expressed specifically in the small intestine is the LPH gene (lactase-phlorizin hydrolase). Genes that are preferentially or exclusively expressed within the lung include an isoform of renin and the calcitonin receptor-like protein.

Genes that are preferentially or exclusively expressed within the kidney include the following: renin, LFB3, vacuolar H(+)-ATPase subunit B, isoform H057, CIC-6c, Ksp-cadherin, CLC-K1, kidney androgen-regulated protein, sodium-phosphate cotransporter, renal cytochrome P-450, parathyroid hormone receptor, and KSP32.

Other genes known to be preferentially or exclusively expressed within certain tissue types include the following: Oct-4, Oct-3, Rex1, SPARC, Brachury, goosecoid, Sox1, beta major globin, Collagen II adult type, Nurr1, Pitx3, keratin, c-kit, stem cell factor, epo, IL3, IL3 receptor, fgf5, nodal, Nkx2.5, EKLF, Msx3, Cdx2, P11, Esrrb, Mash2, Pou5f1, Otx 1, Ebaf, Upp, S1c2a3, Fgf4, H19, and Sox2.

Several different methods for analyzing increases in gene expression are well known in the art. Those amenable to the particular conditions of the methods described herein can be adapted for use in the invention. A particularly preferred method for analyzing gene expression in cells in subcultures being screened in accordance with the invention is mRNA analysis. Examples of direct mRNA analysis methods that might be used include Northern Blotting, dot-blotting, and slot-blotting. Such blotting techniques include the steps of isolating RNA (total cellular RNA or mRNA) from the cells in individual subcultures, immobilizing the isolated RNA on a substrate, and probing the substrate with a labeled probe that specifically binds the RNA species of interest (e.g., the mRNA encoding the gene whose expression is being analyzed). mRNA analysis can also be performed indirectly by, e.g., RT-PCR analysis, wherein cellular mRNA is used to produce cDNA, and sets of gene specific primers are used to amplify specific cDNAs into detectable products. PCR primers for amplifying particular cDNAs corresponding to particular mRNAs can be designed according to known methods based on the known nucleic acid sequences of the particular genes of interest. For high throughput screening, assays using multi-well culture plates and RT-PCR are preferred. In addition, gene chip technology might be used. See, e.g., U.S. Pat. Nos. 6,287,850; 6,262,216; 5,571,639; and 5,143,854.

In addition to analyzing mRNA, other methods for assessing increased gene expression are known. In particular, among these is measuring increased levels of proteins produced by the mRNA of interest (e.g., by antibody-based methods such as ELISA, RIA, immunofluorescence analysis, and flow cytometric analysis; as well as methods that measure enzymatic activity of proteins). Increases in mRNA translation or stability might also be assessed.

The foregoing methods are described in more detail in methodology treatises such as Sambrook et al., supra; and Basic Methods in Molecular Biology, 2nd ed., ed. Davis et al., Appleton and Lange, Norwalk, Conn., 1994.

### EXAMPLES

The present invention is further illustrated by the following specific examples. The examples are provided for illustration only and are not to be construed as limiting the scope or content of the invention in any way.

### Example 1

Screening Assay Using Murine Embryonic Stem Cells

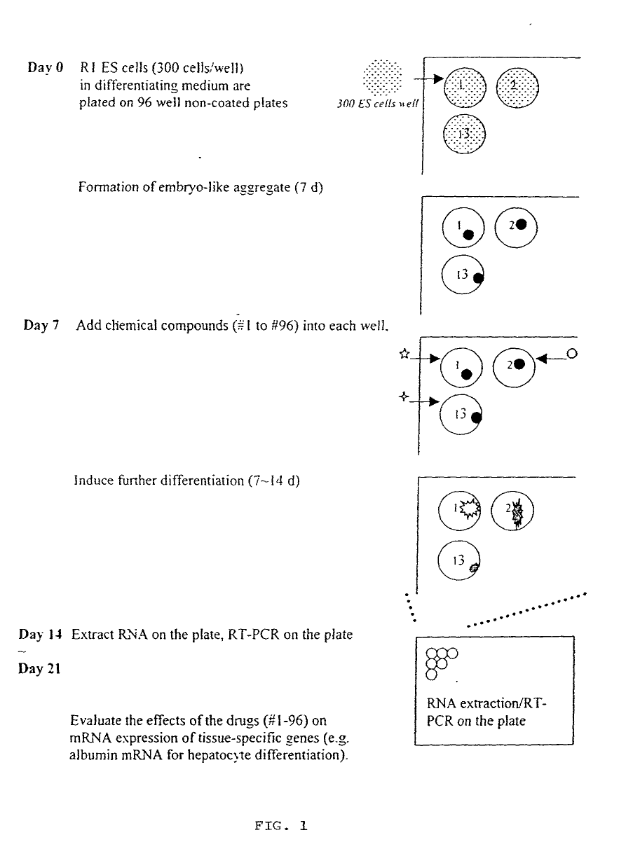

An overview of one method of the invention is presented in FIG. 1. 300 mouse ES cells in differentiating medium are added to wells of a 96 well microtiter plate. The plate is cultured for seven days under conditions that promote differentiation of ES cells. Ninety-six different substances from a library (e.g., a chemical compound library) are then separately added, one substance to a well, to the wells of the plate. The plate is returned to culture for an additional 7-14 days. After this period, total RNA is extracted from each well of the plate. The extracted RNA is then evaluated for increased expression of tissue specific mRNAs (e.g. alpha cardiac myosin-heavy chain mRNA for cardiac myocyte-specific differentiation, albumin mRNA for hepatocyte-specific differentiation, etc.). Methods for evaluating mRNA expression include RT-PCR, dot-blot, and cDNA/gene chip technology.

### Example 2

Induction of Differentiation Of Embryonic Stem Cells Into Hepatocytes

The potential of mouse ES cells to differentiate into hepatocytes in vitro was investigated as described in Hamasaki et al., FEBS lett, 18:497(1):15-19, 2001.

Materials and Methods

Cell Culture—The ES cell lines R1 (129Sv strain), W9.5 (129Sv), and SEK1 null (established from W9.5) (Ganiatsas et al., (1998) Proc. Natl. Acad. Sci. USA 95, 6881-6) were maintained undifferentiated in gelatin-coated dishes in DMEM (GIBCO BRL, Grand Island, N.Y.) containing 15% fetal bovine serum (Atlanta biologicals, Norcross, Ga.), 2 mM L-glutamine, 100 units/ml penicilin, 100 μg/ml streptomycin, 25 mM Hepes (GIBCO BRL), 300 μM monothioglycerol (Sigma, St. Louis, Mo.), and 250 unit/ml recombinant mouse LIF (ESGRO, CHEMICON, Temecula, Calif.). To induce differentiation, ES cells were suspended in IMDM containing 2 mM L-glutamine, 100 units/ml penicillin, 100 μg/ml streptomycin (GIBCO BRL), 20% fetal bovine serum (Atlanta biologicals) and 300 μM monothioglycerol (Sigma). Cells were cultured for 2 days by the hanging-drop method (1×10³ ES cells per 30 μl in each drop) (Metzger et al., (1994) J. Cell. Biol. 126, 701-11). EBs in hanging drops were transferred to suspension culture in 100-mm petri dishes and cultured for an additional 3 days. The resulting EBs were plated onto six-well tissue culture dishes coated with or without Vitrogen (collagen type I) (COHESION, Palo Alto, Calif.). In some experiments, the growth factors were added into culture medium (100 ng/ml acidic fibroblast growth factors (aFGF), 20 ng/ml hepatocyte growth factor (HGF), 10 ng/ml oncostatin M, with 10⁻⁷ M dexamethasone (Sigma), and ITS (5 mg/ml insulin, 5 mg/ml transferrin, 5 μg/ml selenious acid, Collaborative Biomedical Products, Benford, Mass.)).

RT-PCR-Total RNA was extracted using an RNA aqueous kit (Ambion Inc. Austin, Tex.). cDNA was synthesized from 2 μg total RNA using the SuperScript II first-strand synthesis system with oligo (dT) (GIBCO BRL). PCR was performed using Taq DNA polymerase (Eppendorf, Westbury, N.Y.) (94° C., 1 min; specific annealing temperature below, 1 min; 72° C., 1 min). Primers were synthesized for the following mouse genes (oligonucleotide sequences are given in brackets in the order of antisense-, sense-primer followed by the annealing temperature and cycles used for PCR, length of the amplified fragment): transthyretin (5′-CTCACCACAGATGAGAAG (SEQ ID NO: 1), 5′-GGCTGAGTCTCTCAATTC (SEQ ID NO: 2); 55° C.; 25cycles; 225bp), alpha-fetoprotein (5′-TCGTATTCCAACAGGAGG (SEQ ID NO: 3), 5′-AGGCTTTTGCTTCACCAG (SEQ ID NO: 4); 55° C.; 25cycles; 173 bp), alpha 1-antitrypsin (5′-AATGGAAGAAGCCATTCGAT (SEQ ID NO: 5), 5′-AAGACTGTAGCTGCTGCAGC (SEQ ID NO: 6); 55° C.; 30cycles; 484 bp), albumin (5′-GCTACGGCACAGTGCTTG (SEQ ID NO: 7), 5′-CAGGATTGCAGACAGATAGTC (SEQ ID NO: 8); 55° C.; 25cycles; 260 bp), G6P (5′-CAGGACTGGTTCATCCTT (SEQ ID NO: 9), 5′-GTTGCTGTAGTAGTCGGT (SEQ ID NO: 10); 55° C.; 30cycles; 210 bp), TAT (5′-ACCTTCAATCCCATCCGA (SEQ ID NO: 11), 5′-TCCCGACTGGATAGGTAG (SEQ ID NO: 12); 50° C.; 30cycles; 206 bp), beta-actin (5′-TTCCTTCTTGGGTATGGAAT (SEQ ID NO: 13), 5′-GAGCAATGATCTTGATCTTC (SEQ ID NO: 14); 55° C.; 20cycles; 200 bp), SEK1 (5′-TGTATGGAGCTCATGTCTACC (SEQ ID NO: 15), 5′-GTCTATTCTTTCAGGTGCCA (SEQ ID NO: 16); 50° C.; 30cycles; 300 bp).

For each gene, the DNA primers were originated from different exons to ensure that the PCR product represented the specific mRNA species and not genomic DNA. Relative quantitation of albumin gene was performed by ABIPRISM5700 sequence detection system and SYBR green PCR master mix (PE Biosystems, Foster City, Calif.). Beta-actin was used as the endogenous control.

JNK Activity—JNK activity in cell lysates was measured by immunecomplex protein kinase assays using the substrate glutathione S-transferase (GST)- c-Jun (1-79) fusion protein (Minden et al., (1994) Science 266, 1719-23; Ishizuka et al., (1999) J. Immunol. 162, 2087-94). Cell lysates were incubated 30 min with GST-c-Jun (1-79) fusion proteins immobilized on glutathione-Sepharose beads to precipitate JNKs. These beads were resuspended in 50 μl of kinase buffer (20 mM HEPES, 20 mM β-glycerophosphate, 1 mM dithiothreitol, 50 βM Na₃VO₄, and 10 mM MgCl₂, 10 μCi ³²P-γATP). The kinase reaction was performed at 30° C. for 20 min, and stopped by adding SDS sample buffer. The samples were resolved in a SDS gel. The gel was stained with Coomassie blue solution for 5 min and destained, then air dried. Phosphorylated GST-c-Jun was visualized by autoradiography.

Results

In vitro ES Differentiation to Hepatic Lineage—To assess the level of endodermal and hepatic differentiation, the mRNA expression of endodermal—and liver-specific genes was examined. Transthyretin and alpha 1-antitrypsin represent endodermal or yolk-sac-like differentiation and are expressed throughout liver maturation. Alpha-fetoprotein is a marker of the endodermal differentiation as well as an early fetal hepatic marker, and its expression decreases as the liver develops into adult phenotype. Expression of albumin, the most abundant protein synthesized by mature hepatocytes, starts in early fetal hepatocytes (E12) and reaches the maximal level in adult hepatocytes. Although albumin is known to be a hepatocyte differentiation marker, it is also expressed weakly in yolk sac. At a late gestational or perinatal stage, glucose 6 phosphatase (G6P) is predominantly expressed in the liver. Tyrosine aminotransferase (TAT) represents an excellent enzymatic marker for peri—or postnatal hepatocyte-specific differentiation. These enzymes are not synthesized in significant quantities prior to birth but are rapidly activated early in the neonatal developmental period. Since hormone-regulated TAT activity is strictly limited to the parenchymal cells of the adult liver, it has been used extensively for monitoring cellular differentiation in experimental models for liver development/maturation in vitro.

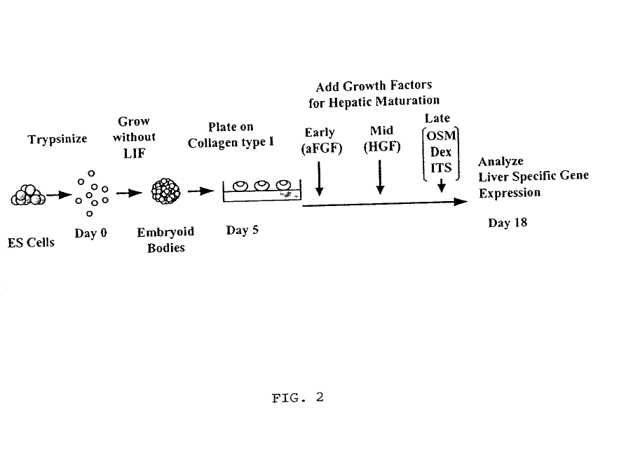

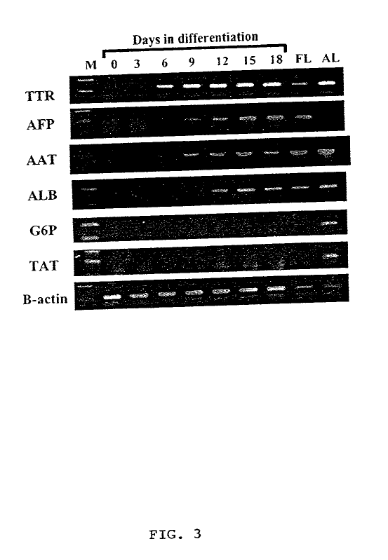

Undifferentiated ES cells did not express these endodermal or hepatocyte lineage genes—FIG. 2 depicts the in vitro ES differentiation procedure used in this study. FIG. 3 illustrates the pattern of endodermal specific gene expression in differentiating EBs without additional growth factors. Transthyretin was expressed within 6 days after removal of the LIF. Alpha-fetoprotein and alpha 1-antitrypsin were expressed within Day 9. Albumin mRNA expression first appeared within Day 12. Late differential markers of hepatocyte, TAT and G6P were not detectable throughout the time course (up to Day 18). These data indicate that ES cells spontaneously differentiate toward hepatic or yolk sac lineage cells, but they do not differentiate into mature hepatocytes.

Induced Hepatic Maturation In vitro—During embryonic development of mice, the initial event of liver ontogeny occurs on embryonic day 9 (E9). In this early stage, FGFs, derived from adjacent cardiac mesoderm, commit the foregut endoderm to forming the liver primodium. Over the next two days, the liver bud proliferates and migrates into surrounding septum transversum, which consists of loose connective tissue containing collagen. Hepatic precursors are in direct contact with connective tissue matrix. During and after mid stage of hepatogenesis, surrounding mesenchymal cells secrete HGF and support fetal hepatocytes. Indeed, in mice genetically lacking HGF, the embryonic liver is reduced in size and shows extensive loss of parenchymal cells. From E12 through E16, the fetal liver becomes the major site for hematopoiesis. During this late stage, hematopoietic cells produce oncostatin M that induces maturation of murine fetal hepatocytes.

Based on these previous reports for embryonic liver development, growth factors and cell culture matrix were applied to induce hepatic maturation of EBs in vitro (FIG. 2). Initially EBs were attached to collagen coated culture plates at Day 5 in vitro differentiation. As an early stage factor potentially inducing hepatic differentiation, aFGF was added from Day 9 to Day 12. From Day 12 to Day 18, HGF was added as a mid-stage factor. Oncostatin M (OSM), dexamethasone (Dex) and a mixture of insulin, transferrin and selenious acid (ITS) were added as late-stage factors from Day 15 to Day 18. The patterns of hepatic lineage gene expression were analyzed at Day 18.

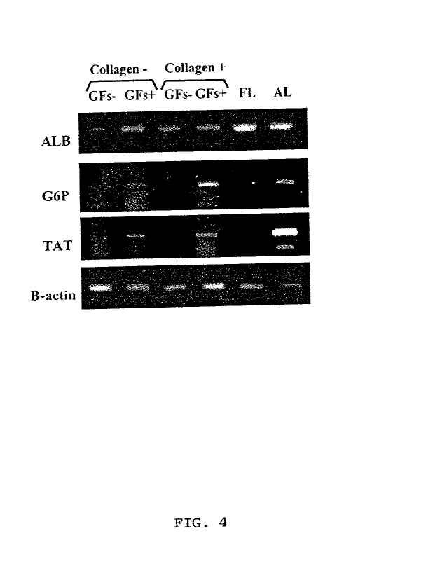

As shown in FIG. 4, a combination of these growth factors enhanced the expression of albumin mRNA, which is an indicator of hepatocyte maturation. The expression of albumin was increased 9.5-fold and 7.4-fold (real-time PCR) by the growth factors on collagen-uncoated culture and collagen-coated culture, respectively. Moreover, G6P and TAT genes, indicators of hepatocyte maturation, were now expressed in EBs in the presence of the growth factors. It appeared that collagen coating further enhanced the expression of G6P and TAT.

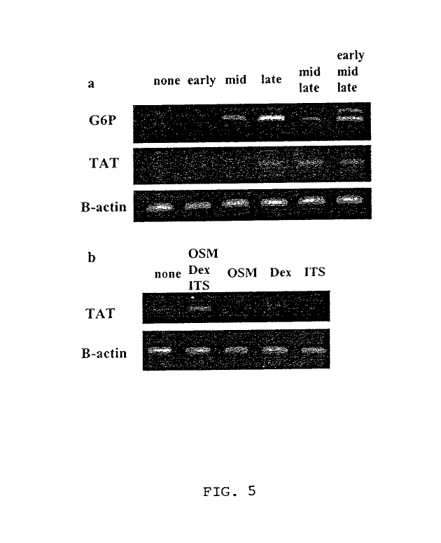

The effects of growth factors at individual stages on hepatic development using EBs plated on collagen coated dishes were also examined. As demonstrated in FIG. 5a, the mid-stage factor (HGF) or late stage factors (OSM, Dex, ITS) were critical for G6P expression. The late-stage factors exclusively enhanced TAT gene expression. Although Dex, by itself, slightly induced TAT expression, the mixture of the late stage factors mostly enhanced the TAT expression (FIG. 5b).

Maturation of SEK1 Null ES Cells into Hepatic Lineage In vitro—SEK1 (also known as MKK4 and JNKK) is a member of mitogen-activated protein kinase activator family. SEK1 deficiency leads to an embryonic lethality between E10.5 and E12.5 and is associated with abnormal liver development. SEK1 null fetal mice show that the visceral endoderm normally develops into primordial liver, but parenchymal hepatocytes undergo massive apoptosis. This phenomenon indicates the SEK1 signaling pathway is exclusively required after a certain period of early hepatogenesis. Because of its embryonic lethality, it is hard to further assess the role of SEK1 in the late stage hepatogenesis in vitro.

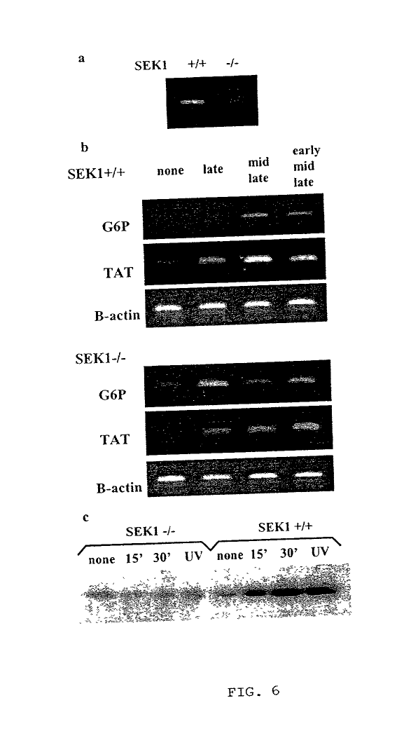

The potential of SEK1 null ES cells to differentiate into mature hepatocytes was investigated using the in vitro ES cell differentiation system described above. FIG. 6a showed the expression of SEK1 mRNA in differentiated EBs (Day 18). SEK1 mRNA was not detected in SEK1 null EBs. The expression of TAT and G6P mRNA was induced in SEK1 null EBs by late stage growth factors as well as in control wild type EBs (FIG. 6b). The late stage growth factors induced JNK activity, a downstream kinase of SEK1, in wild type EBs but not in SEK1 null EBs (FIG. 6c). These data indicated that the SEK1 signaling pathway is not indispensable in late stage maturation of hepatocytes.

### Example 3

Increases in Liver-Specific Gene Expression in Differentiating ES Cells

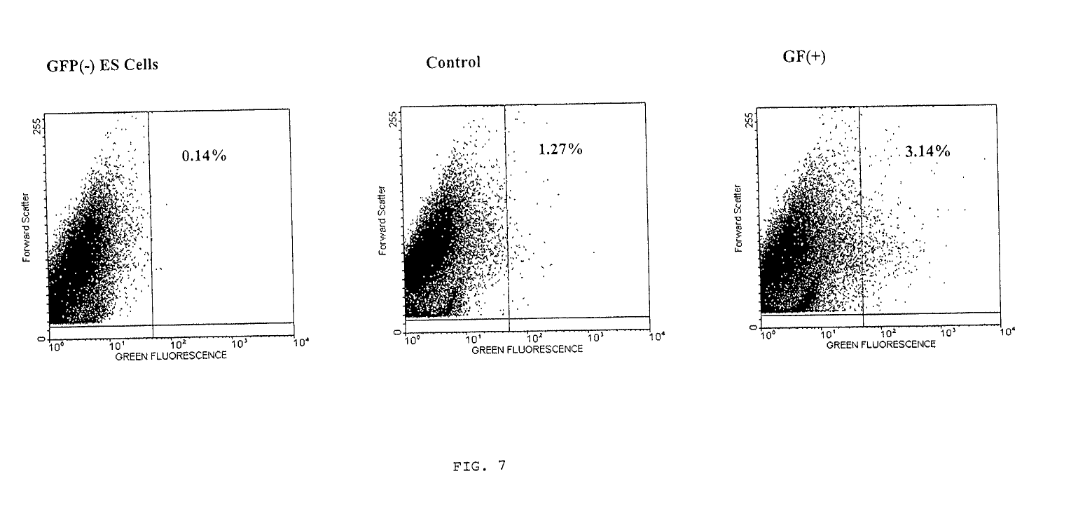

Referring now to FIG. 7, liver-specific gene expression was analyzed by flow cytometry of differentiating ES cells. A combination of the growth factors (as in Example 2) was added to a culture of murine ES cells expressing green fluorescent protein (GFP) under the control of the hepatocyte-specific promoter (i.e., alpha-fetoprotein promoter). These ES cells were subjected to culture conditions that promote in vitro differentiation with (GF+) or without (Control) the growth factors as described above. Cells were harvested at Day 18, and treated with collagenase. Single suspended cells were subjected to flow cytometric analysis to count GFP positive cells. As demonstrated in the histograms of FIG. 7, addition of the growth factors increased the population of GFP positive hepatic cells in the cultures. The far left panel represents the analysis of parental R1 ES cells with no GFP vector transfected.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a drug candidate for promoting tissue-specific differentiation of a stem cell, the method comprising the steps of:
(A) providing a library of test substances, the library comprising at least a first test substance and a second test substance, the first and second test substances having different molecular structures;
(B) providing an in vitro culture of stem cells, the culture being divided into at least a first subculture and a second subculture;
(C) contacting the first subculture with the first test substance and the second subculture with the second test substance;
(D) culturing the first and second subcultures respectively contacted with the first and second test substances under conditions that would promote tissue-specific differentiation of the stem cells if an agent that promoted tissue-specific differentiation was in contact with the stem cells; and
(E) analyzing the cells in the first and second subcultures for increased tissue-specific gene expression.

2. The method of claim 1, wherein the stem cells are embryonic stem cells.

3. The method of claim 2, wherein the embryonic stem cells are mammalian embryonic stems cells.

4. The method of claim 3, wherein the mammalian embryonic stem cells are murine embryonic stems cells.

5. The method of claim 4, wherein the murine embryonic stem cells R1 embryonic stems cells.

6. The method of claim 3, wherein the mammalian embryonic stem cells are human embryonic stems cells.

7. The method of claim 1, wherein the conditions that would promote tissue-specific differentiation of the stem cells comprises culturing the first and second subcultures in a differentiating medium.

8. The method of claim 1, wherein the conditions that would promote tissue-specific differentiation of the stem cells comprises culturing the first and second subcultures at about 37° C.

9. The method of claim 1, wherein the conditions that would promote tissue-specific differentiation of the stem cells comprises culturing the first and second subcultures in a humidified, carbon-dioxide containing incubator.

10. The method of claim 1, wherein the conditions that would promote tissue-specific differentiation of the stem cells comprises culturing the first and second subcultures for a time period of at least five days.

11. The method of claim 10, wherein the time period is at least seven days.

12. The method of claim 11, wherein the time period is between seven and eighteen days.

13. The method of claim 1, wherein the first and second subcultures are cultured in a microtiter plate.

14. The method of claim 1, wherein the step (E) of analyzing the cells in the first and second subcultures for increased tissue-specific gene expression comprises isolating mRNA from the first and second subcultures.

15. The method of claim 14, wherein total cellular RNA is isolated from the first and second subcultures.

16. The method of claim 14, wherein the step (E) further comprises reverse-transcribing the mRNA to create cDNA.

17. The method of claim 1, wherein the step (E) of analyzing the cells in the first and second subcultures for increased tissue-specific gene expression comprises performing a polymerase chain reaction (PCR).

18. The method of claim 14, wherein the isolated mRNA is immobilized on a substrate.

19. The method of claim 18, wherein the substrate is contacted with a probe that specifically hybridizes to the tissue-specific mRNA.

20. The method of claim 1, wherein the step (E) of analyzing the cells in the first and second subcultures for increased tissue-specific gene expression is performing using gene chip technology.
